(19) United States
(12) Patent Application Publication
(10) Pub. No.: US 2004/0034547 A1
(43) Pub. Date: Feb. 19, 2004

(54) Medical device which can be subordinated to a planning medical system and planning medical system which can be made superordinate to a medical device

(76) Inventors: Thomas Birkhoelzer, Radolfzell (DE); Frank Krickhahn, Herzogenaurach (DE); Juergen Vaupel, Weisendorf (DE)

Correspondence Address: HARNESS, DICKEY & PIERCE, P.L.C., P.O.BOX 8910, RESTON, VA, 20195, US

(21) Appl. No.: 10/389,908

(22) Filed: Mar. 18, 2003

(30) Foreign Application Priority Data
Mar. 18, 2002 (DE) 10211950.3

(51) Int. Cl. G06F 17/60; A61B 5/00

(52) U.S. Cl. 705/2; 600/300

(57) ABSTRACT

A medical device includes a storage device in which descriptions of examination orders which can be executed can be stored. The examination orders can be transferred to a planning medical system via a read-out interface. A selection of an examination order can be transferred by the planning medical system via an instruction-presetting interface. The selection can be received by the medical device via an instruction transfer interface and a corresponding job order can be executed.

The present application hereby claims priority under 35 U.S.C. §119 on German patent application number DE 10211950.3 filed Mar. 18, 2002, the entire contents of which are hereby incorporated herein by reference.

### FIELD OF THE INVENTION

The present invention generally relates to a medical device which can be subordinated to a planning medical system having an instruction transfer interface. Preferably, it is possible for a selection of an examination order to be received by the medical device from the planning medical system via the instruction transfer interface, and a procedure which corresponds to the examination order selected to be executed. The present invention also generally relates to a corresponding planning medical system which can be made superordinate to the medical device.

### BACKGROUND OF THE INVENTION

Such medical devices and planning medical systems are generally known. In them, the term “examination order” is used for a predetermined sequence of steps which is executed by a medical device when the examination order is selected. The medical device can be embodied, for example, as a medical device which executes the examination order itself or as a medical system which passes on the examination order to a medical device, for example as what is referred to as an RIS (Radiology Information System) or as a CIS (Cardiology Information System). The superordinate planning medical system is typically an RIS, a CIS or an HIS (Hospital Information System).

In the typical workflow of radiology, the examination steps which are to be carried out on a patient are defined by use of the planning system. The starting point here is the order of the attending physician. For example, part of a patient's body is to be examined for a growth (cancer). This order must then be converted into specific procedures or examination orders, for example execution of an X-ray scan in the computer tomograph using specific parameters. The examination orders are in turn generally composed of a plurality of individual working steps.

The selection of the examination order to be executed is transmitted—usually in the DICOM format—as an SPS (scheduled procedure step) from the planning medical system to the medical device and the examination order is then carried out by the latter. A list of the examination orders which can be executed is generally available to the planning medical system for this purpose. An operator of the planning system simply has to select one of the examination orders which can be executed.

The examination orders are highly device-dependent in their configuration. They are therefore typically defined on the medical devices which then also execute these examination orders themselves. The definition of the working steps on the respective medical devices is possible using specific setup functions.

If a new procedure is defined on the medical device, this procedure must be made known to the planning medical system in some form in order to be able to selected. In the prior art, this is generally carried out by manual inputting of a description of the examination order into the planning system by a system operator. The system operator manages both the examination orders which can be executed by the medical device which can be subordinated and the descriptions of the examination orders which are stored in the superordinate planning medical system.

If such an examination order list is of low complexity, this procedure may be extremely efficient. However, a large amount of work is involved if there are relatively high levels of complexity with, for example, 10 to 20 examination orders per medical device, a plurality of medical devices and one to two changes per month and examination order. Moreover, a considerable amount of expenditure is necessary in order to continuously ensure consistency between the examination orders stored in the various units and their descriptions.

### SUMMARY OF THE INVENTION

An object of an embodiment of the present invention is to ensure in a simple, rapid, convenient and in particular also failsafe way that the descriptions of the examination orders of the subordinate medical device which can be executed are known to the superordinate planning system.

An object may be achieved with respect to the medical device in one embodiment, that it has a storage device and a read-out interface. It is possible to store descriptions of examination orders which can be executed by the medical device in the storage device. Further, it is possible to read out the descriptions from the storage device and transfer them to the planning medical system via the read-out interface.

In a corresponding way, an object may be achieved with respect to a superordinate planning system by use of a read-in interface via which the planning medical system can receive descriptions, transferred from the medical device, of examination orders which can be executed by the medical device.

If the communication between the planning system and the medical device, that is to say in particular the transfer of the descriptions and of the selection, is carried out in the DICOM format, a proven, virtually standardized method can be used.

If the descriptions in the storage device are stored in the XML format, the descriptions are restricted in a self-explanatory way.

If changes and newly preset values of descriptions are transferred automatically to the planning system, it is always ensured that the planning medical system knows the current descriptions. Alternatively, it would be possible for the transfer of the descriptions to the planning system to be capable of being initiated by the planning system. If this is the case, the initiation is preferably repeated at periodic time intervals.

If the descriptions each have a name for the examination order, the operator control of the planning medical system is particularly user-friendly.

If the descriptions each include an indication, a billing key, an update status and/or an issuer indication, the use is still user-friendly.

If the descriptions of the planning system have place markers to be filled with contents, the examination orders can be handled even more flexibly by the planning system. This applies in particular if the planning system handles the process of filling with contents interactively.

The medical device can execute the examination order itself. Alternatively, it may be embodied as a medical system which passes on the examination order to a medical device.

If a user can interactively preset a selection instruction to the planning medical system via an input device, the selection is made particularly easily. If, in the process, the descriptions can be output to the user as a list via an output device, the selection can be made in a particularly clearly organized way.

### BRIEF DESCRIPTION OF THE DRAWINGS

Further advantages and details emerge from the following description of an exemplary embodiment. In the drawings, in each case in a basic view,

FIG. 1 shows a structure of a plurality of medical devices,

FIG. 2 shows a view of an examination order,

FIG. 3 shows a list with examination orders, and

FIGS. 4 and 5 are flowcharts.

### DETAILED DESCRIPTION OF THE PREFERRED EMBODIMENTS

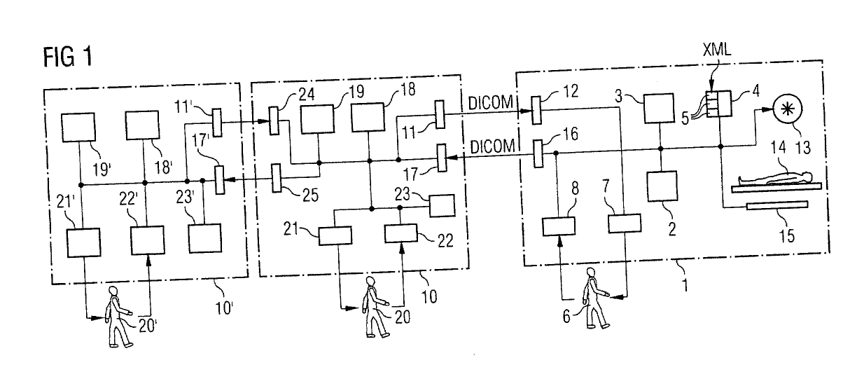

According to FIG. 1, a structure of medical devices firstly has a mechanical device 1, for example an X-ray system 1. The medical device 1 is computer-controlled. It therefore has a main unit 2 which processes a control program 3. For this purpose, examination orders 5 are stored in a storage device 4. The examination orders 5 can be read out individually from the storage device 4 by an operator 6 by use of an output device 7 and can be represented in a way which is easy for the operator 6 to read. The called examination order 5 can then be modified, if appropriate also newly preset, by means of an input device 8, and stored—for the first time or again—in the storage device 4. The storage in the storage device 4 is carried out here in the XML format, as indicated by the letters “XML” in FIG. 1.

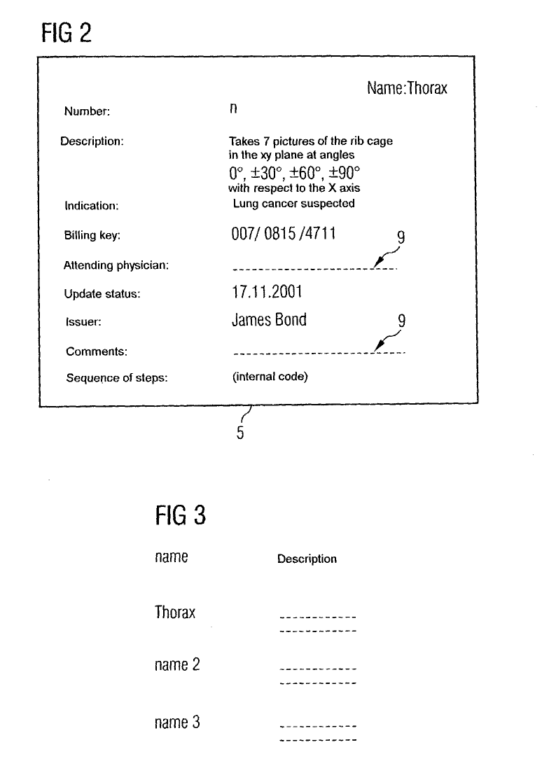

According to FIG. 2, the examination orders 5 have the following structure:

In a first field they contain a number n. The examination orders 5 are managed internally using this number n. In the next field there is a name, according to FIG. 2 the designation “Thorax”. The examination order 5 can be called by the operator 6 using this name. The examination order 5 is then described in a third field. According to FIG. 2, the examination order 5 executes, for example, seven photographs of the rib cage in the xy plane at angles of 0°, +/−30°, +/−60° and +/−90° with respect to the x axis.

The next field then contains a specific indication, i.e. clinical pictures in which this examination order 5 could typically be applied. According to FIG. 2, this contains the entry “lung cancer suspected”. A billing key which can be used to produce a bill by machine is then stored in the next field. The subsequent field, the attending physician, is empty. A space marker 9, which is to be filled later with contents, is arranged at this point.

The subsequent fields then contain an update status, for example version as at 17.11.2001, the issuer of the examination order 5 and comments. The comments may also be provided in the form of a space marker 9.

The specific sequence of steps to be executed by the medical device 1 is then stored in the last region. An internal code which has to be known only by the medical device 1 itself is located here.

Each such examination order 5 can be selected from a planning medical system 10, for example by presetting the number n or the name. The selection is carried out by here by means of an instruction-presetting interface 11 of the planning medical system 10. The planning medical system 10 is embodied, for example, as an RIS (Radiology Information System). The selection is transferred here in the DICOM format, as indicated by the letters “DICOM” in FIG. 1.

In this case, the medical device 1 receives the selection via an instruction transfer interface 12 and executes the corresponding examination order 5 (itself). Owing to the execution of the examination order 5 which is provided with the selection, the medical device 1 is a device which is subordinated to the planning system 10. This is in turn made superordinate to the medical device 1.

During the execution of the examination order 5, an X-ray device 13, for example, is actuated so that the photographs of a part of the body of a patient 14 (illustrated only schematically) which are specified in accordance with the selected examination order 5 are taken and read into the medical device 1 by a recording medium 15.

In order to be able to use the planning medical system 10 to select an examination order 5 which is to be executed by the medical device 1, the examination orders 5 must of course be known to the planning medical system 10. For this purpose, the medical device 1 has a read-out interface 16 which is connected to a read-in interface 17 of the planning medical system 10. The examination orders 5 which can be executed can be read out from the storage device 4 and transferred to the planning medical system 10 via the read-out interface 16 and the read-in interface 17—with the exception of the specific sequence of steps which is significant only for the medical device 1. The system 10 receives the examination orders 5, in particular their names and descriptions. Here too, communication takes place between the medical device 1 and the planning medical system 10 in the DICOM format, as indicated by the letters “DICOM”.

The planning medical system 10 also has a main unit 18 which executes a control program 19. During the processing of the control program 19, a list, inter alia, of the examination orders 5 which can be executed (illustrated by way of example in FIG. 3) can be output to an operator 20 of the planning medical system 10 by the main unit 18. Here, according to FIG. 3, only the names and the descriptions of the examination orders 5 are placed in the list. The outputting is carried out by way of an output device 21, for example a monitor, or a similar display device. A customary input device 22 (keypad, mouse) can then be used by the operator 20 to select interactively one of the examination orders 5 which can be executed. This selection constitutes a selection instruction so that the planning medical system 10 transfers the corresponding selection to the medical device 1.

The examination orders 5 which can be executed are stored within the planning medical system 10 in a storage device 23. It is therefore also possible—as an alternative to the transfer of the selection to the medical device 1—for the selected examination order 5—with the exception of the internal code of the examination order 5—to be represented completely by means of the output device 21. In this case, it is, in particular, possible for the operator 20 of the planning system 10 to fill the place markers 9 of the examination orders 5 with contents by use of interactive preset values.

The transfer of the examination orders 5, which can be executed, by the subordinate medical device 1 to the superordinate planning medical system 10 can essentially be carried out on an event-controlled or time-controlled basis.

In the aforementioned case, the control program 3 of the medical device 1 is embodied in such a way that although it receives changes and new entries of examination orders 5 in a step 26 it also stores them in a step 27. The execution of step 28 is forcibly brought about owing to the storage in step 27. In this step 28, all the examination orders 5, or at least the newly stored ones, are automatically transferred to the superordinate planning medical system 10. This procedure is illustrated schematically in FIG. 4.

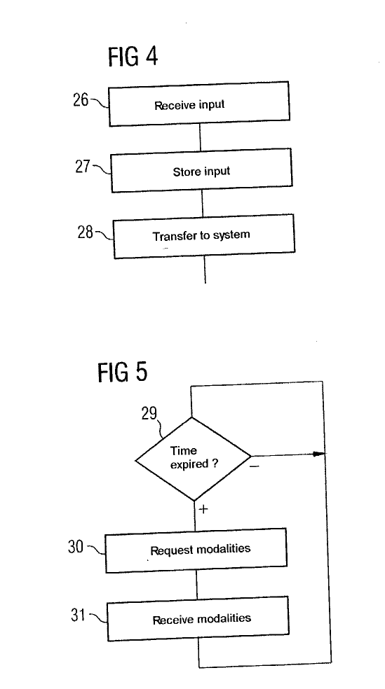

Alternatively, according to FIG. 5, it is possible for the superordinate planning medical system 10 to repeatedly check, in a step 29 of its control program 19, whether a timer has expired. If this is the case, it initiates, in a step 30, transfer of the examination orders 5 of the medical device 1 to the planning system 10 and receives these examination orders 5 in a step 31. The initiation of the transfer of the modalities 5 is repeated at periodic time intervals by monitoring the timer for expiry of the time.

In terms of the interaction between the planning medical system 10 and the medical device 1, the former is the superordinate planning medical system and the latter the subordinate medical device. However, it is possible, as indicated in FIG. 1, to make a further planning medical system 10′ superordinate to the planning medical system 10. The further planning medical system 10′ has the same components as the first planning medical system 10. The latter are merely additionally provided with a prime mark in order to distinguish them. In this case, the first planning medical system 10 additionally has an instruction transfer interface 24 and a read-out interface 25.

In terms of the interaction between the two planning medical systems 10, 10′, the planning medical system 10 is now the subordinate medical device. If an examination order 5 is then selected by the superordinate planning medical system 10′, the planning medical system 10 does not carry out the corresponding job order itself but rather passes it on to the medical device 1.

The elements according to the invention which are described above ensure, in a simple and particularly consistent way, that the planning system 10 or 10′ always has available the examination orders 5 which can be executed by the medical device 1.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. A medical device adapted to be subordinated to a planning medical system, comprising:
a storage device adapted to store descriptions of examination orders executable by the medical device;
a read-out interface adapted to read out the descriptions from the storage device and transfer them to the planning medical system; and
an instruction transfer interface, wherein the medical device is adapted to receive a selection of an examination order from the planning medical system via the instruction transfer interface, and wherein a job order corresponding to the selected examination order is adapted to be executed.

2. The device as claimed in claim 1, wherein the descriptions are adapted to be transferred to the planning system in the DICOM format.

3. The device as claimed in claim 1, wherein the selection is adapted to be received in the DICOM format.

4. The device as claimed in claim 1, wherein the descriptions are adapted to be stored in the storage device in the XML format.

5. The device as claimed in claim 1, wherein changes and newly preset values of descriptions are automatically transferred to the planning system.

6. The device according to claim 1, wherein the descriptions each have a name for the examination order.

7. The device according to claim 6, wherein descriptions each contain at least one of an indication, a billing key, an update status and an issuer indication.

8. The device as claimed in claim 6, wherein the descriptions of the planning system include place markers to be filled with contents.

9. The device as claimed in claim 1, the device executes the job order to be executed.

10. The device as claimed in claim 1, wherein the device is embodied as a medical system which passes the job order to be executed onto a medical device.

11. A planning medical system, adapted to be superordinate to a medical device, comprising:
a read-in interface; and
an instruction-presetting interface, wherein the planning medical system is adapted to receive descriptions, transmitted by the medical device via the read-in interface, of examination orders executable by the medical device, and wherein a selection of one of the examination orders is adapted to be transferred by the planning medical system via the instruction-presetting interface of the medical device.

12. A system as claimed in claim 11, wherein the descriptions transferred by the medical device are adapted to be received in the DICOM format.

13. The system as claimed in claim 11, wherein the selection of the examination order is adapted to be transferred in the DICOM format.

14. The system as claimed in claim 11, wherein a selection instruction is adapted to be preset to said system by a user via an input device.

15. The system as claimed in claim 14, wherein the descriptions are adapted to be output to the user as a list via an output device.

16. The system as claimed in claim 11, wherein the transfer of the descriptions to the planning system is adapted to be initiated by the planning system.

17. The system as claimed in claim 16, wherein the initiation of the transfer of the descriptions is repeated at periodic time intervals.

18. The system as claimed in claim 11, wherein place markers of the descriptions are adapted to be filled with contents.

19. The system as claimed in claim 18, wherein the contents are adapted to be preset interactively to the planning system.

20. The device as claimed in claim 7, wherein the descriptions of the planning system include place markers to be filled with contents.

21. The device as claimed in claim 2, wherein the selection is adapted to be received in the DICOM format.

22. The system as claimed in claim 12, wherein the selection of the examination order is adapted to be transferred in the DICOM format.

23. The system as claimed in claim 12, wherein a selection instruction is adapted to be preset to said system by a user via an input device.

24. The system as claimed in claim 13, wherein a selection instruction is adapted to be preset to said system by a user via an input device.

25. A medical device adapted to be subordinated to a planning medical system, comprising:
storage means for storing descriptions of examination orders executable by the medical device;
read-out interface means for reading out the descriptions from the storage device and for transferring them to the planning medical system; and
instruction transfer interface, wherein the medical device is adapted to receive a selection of an examination order from the planning medical system via the instruction transfer interface, and wherein a job order corresponding to the selected examination order is adapted to be executed.

26. The device as claimed in claim 25, wherein the descriptions are transferred to the planning system in the DICOM format.

27. The device as claimed in claim 25, wherein the selection is received in the DICOM format.

28. The device as claimed in claim 25, wherein the descriptions are stored in the XML format.
